(19) United States
(12) Patent Application Publication
(10) Pub. No.: US 2024/0041754 A1
(43) Pub. Date: Feb. 8, 2024

(54) NOVEL SACCHAROMYCES CEREVISIAE STRAIN AND USE THEREOF

(71) Applicant: LG HOUSEHOLD & HEALTH CARE LTD., Seoul (KR)

(72) Inventors: Heqing CUI, Seoul (KR); Haemin JUNG, Seoul (KR)

(73) Assignee: LG HOUSEHOLD & HEALTH CARE LTD., Seoul (KR)

(21) Appl. No.: 18/258,841

(22) PCT Filed: Dec. 23, 2021
(86) PCT No.: PCT/KR2021/019700

§ 371 Date: Jun. 22, 2023

(30) Foreign Application Priority Data
Dec. 23, 2020 (KR) 10-2020-0182203

(51) Int. Cl. A61K 8/9728; C12N 1/18; A61Q 19/00; A61Q 19/08

(52) U.S. Cl. CPC A61K 8/9728; C12N 1/185; A61Q 19/007; A61Q 19/08; C12R 2001/845

(57) ABSTRACT

Provided is a Saccharomyces cerevisiae strain deposited under Accession No. KCTC 14268BP; a cosmetic composition, a quasi-drug composition, or a food composition, each including any one or more selected from the group consisting of the strain, a culture thereof, and a fermentation product thereof; a method of improving the skin using the strain, the culture thereof, and the fermentation product thereof; and use of the strain, the culture thereof, and the fermentation product thereof, for the skin improvement.

### TECHNICAL FIELD

The present invention relates to a Saccharomyces cerevisiae strain deposited under Accession No. KCTC 14268BP; a cosmetic composition, a quasi-drug composition, or a food composition, each including any one or more selected from the group consisting of the strain, a culture thereof, and a fermentation product thereof; a method of improving the skin using the strain, the culture thereof, and the fermentation product thereof; and use of the strain, the culture thereof, and the fermentation product thereof, for the skin improvement.

### BACKGROUND ART

Some components of cosmetics often cause various skin troubles such as inflammation, pimples, swelling, etc. (Maibach. H. I., Contact Dermatitis, 6. 369-404, 1980). In addition, various materials used as raw materials of functional cosmetics may also have problems of low safety or insignificant effects on the skin.

For example, retinoids, adenosine, animal placenta-derived proteins, and chlorella extracts are known as cosmetics for enhancing skin elasticity and for improving wrinkles. Retinol, which is the most widely known, has limitations in usage due to safety issues such as irritation, etc., when applied to the skin, and chlorella extract is difficult to expect substantial skin elasticity enhancement and wrinkle improvement effects due to its poor effect.

For another example, commonly known whitening ingredients include kojic acid, arbutin, hydroquinone, vitamin C or derivatives thereof, and various plant extracts. However, there are problems in that when applied to the skin, they have limitations in usage due to safety issues such as irritation, redness, etc., or it is difficult to expect substantial effects due to their poor effects.

### DISCLOSURE

### Technical Problem

The present inventors have conducted research on raw materials from natural resources, which are safer and have excellent skin improvement effects, and they confirmed that Saccharomyces cerevisiae has a skin improvement effect, and in particular, a Saccharomyces cerevisiae strain newly discovered from Dendrobium candidum flowers has a remarkably excellent skin improvement effect, as compared to other Saccharomyces cerevisiae strains, thereby completing the present invention.

### Technical Solution

An object of the present invention is to provide a Saccharomyces cerevisiae strain deposited under Accession No. KCTC 14268BP.

Another object of the present invention is to provide a cosmetic composition for improving the skin, the cosmetic composition including any one or more of the strain, a culture thereof, and a fermentation product thereof.

Still another object of the present invention is to provide a quasi-drug composition for improving the skin, the quasi-drug composition including any one or more of the strain, the culture thereof, and the fermentation product thereof.

Still another object of the present invention is to provide a food composition for improving the skin, the food composition including any one or more of the strain, the culture thereof, and the fermentation product thereof.

Still another object of the present invention is to provide a method of improving the skin using any one or more of the strain, the culture thereof, and the fermentation product thereof.

Still another object of the present invention is to provide use of any one or more of the strain, the culture thereof, and the fermentation product thereof in the skin improvement.

### Advantageous Effects

Since a Saccharomyces cerevisiae strain of the present invention, a culture thereof, or a fermentation product thereof has an excellent skin improvement effect, it is highly applicable as a cosmetic composition or quasi-drug composition for skin improvement. Accordingly, various industrial applications as in cosmetics and quasi-drugs, etc. may be expected.

### DETAILED DESCRIPTION OF PREFERRED EMBODIMENTS

Hereinafter, the present invention will be described in detail as follows. Meanwhile, each description and embodiment disclosed in this disclosure may also be applied to other descriptions and embodiments. Further, all combinations of various elements disclosed in this disclosure fall within the scope of the present invention. In addition, the scope of the present invention is not limited by the specific description described below.

An aspect of the present invention provides a Saccharomyces cerevisiae strain deposited under Accession No. KCTC 14268BP.

As used herein, the “Saccharomyces cerevisiae” refers to a species of yeast belonging to the genus Saccharomyces in the family Saccharomycetaceae. This species has been widely used since ancient times, playing an important role in winemaking, baking, and brewing. Since characteristics of Saccharomyces cerevisiae vary depending on the growth zone and natural environment conditions, the strain and activity of its fermentation product may vary, and Saccharomyces cerevisiae strain of the present invention may be derived from Dendrobium candidum, but is not limited thereto.

As used herein, the “Dendrobium candidum” is one of the Orchidaceae plants widely known as traditional medicinal plants. Since ancient times, the stem of Dendrobium candidum has been widely used for medicinal purposes. It is terete, 15 cm to 50 cm long, 0.15 cm to 0.3 cm in diameter, 1 cm to 4 cm between nodes, and the outer surface is yellow, and part of the lower part is glossy with vertical wrinkles. There are inflorescence stalk scars and remaining leaf sheaths on the node, and the entrance of the leaf sheath is open. As the Dendrobium candidum of the present invention, those commercially available may be purchased and used, or those harvested or cultivated in nature may be used.

The Saccharomyces cerevisiae strain of the present invention may be derived from Dendrobium candidum, and the Dendrobium candidum may be used by selecting a specific flower part among plant parts. Therefore, the Saccharomyces cerevisiae strain may be derived from Dendrobium candidum flowers, but is not limited to the part, origin, and method of obtaining, etc.

Specifically, in one exemplary embodiment of the present invention, it was confirmed that a fermentation product of Dendrobium candidum-derived Saccharomyces cerevisiae (KCTC 14268BP) has an excellent skin improvement effect, as compared to a fermentation product of a general yeast Saccharomyces cerevisiae (KCTC 7296).

Another aspect of the present invention provides a cosmetic composition for improving the skin, the cosmetic composition including any one or more of the Saccharomyces cerevisiae deposited under Accession No. KCTC 14268BP, a culture thereof, and a fermentation product thereof.

The culture of the present invention refers to a culture liquid obtained by culturing a specific microorganism in a culture medium, a concentrated culture liquid, a dry product of the culture liquid, a culture filtrate, a concentrated culture filtrate, or a dry product of the culture filtrate, and the culture liquid refers to those including specific bacteria, and the culture filtrate refers to those substantially not including specific bacteria (which means that specific bacteria to be separated by filtration, etc. are substantially excluded, which does not mean that the bacteria are completely excluded from the filtrate.). The formulation of the culture is not limited, but it may be, for example, a liquid, an emulsion, or a solid.

As used herein, the term “fermentation” refers to a process in which microorganisms break down organic materials using their own enzymes, excluding putrefaction. Fermentation and putrefaction proceed by similar processes, but when useful substances are produced as a result of decomposition, it is called fermentation, and when bad smells or harmful substances are produced, it is called putrefaction.

In the present invention, the method of obtaining the fermentation product from the strain is not particularly limited, and the fermentation product may be obtained according to a method commonly used in the art or similar art.

In the present invention, the fermentation product obtained from the strain includes not only a fermented material itself, but also all types of materials including the fermentation product generated from the strain, such as a culture medium of a strain in which the strain and the culture coexist, a fermentation product obtained by filtering the strain from the culture medium, a fermentation product obtained by sterilizing the strain from the culture medium and filtering the same, an extract obtained by extracting the fermentation product or the culture medium including the same, a dilution obtained by diluting the fermentation product or the extract thereof, a concentrate, a dry product obtained by drying the fermentation product or the extract thereof, a lysate obtained by collecting and disrupting cells of the strain, etc.

In one specific embodiment, the skin improvement may be any one or more selected from the group consisting of skin moisturizing, wrinkle improvement, promotion of hyaluronic acid production, promotion of collagen synthesis, elasticity enhancement, and anti-aging, but is not limited thereto.

As used herein, the term “improvement” refers to all of the actions that at least reduce parameters related to the relief or treatment of conditions, for example, severity of symptoms, and may include any one or more of skin moisturizing, wrinkle improvement, promotion of hyaluronic acid production, promotion of collagen synthesis, elasticity enhancement, and anti-aging.

Specifically, the present inventors confirmed that the fermentation product of Saccharomyces cerevisiae KCTC 14268BP has an excellent skin improvement effect, as compared to a fermentation product of a general yeast Saccharomyces cerevisiae KCTC 7296. More specifically, the present inventors confirmed that the fermentation product of Saccharomyces cerevisiae KCTC 14268BP has excellent effects of enhancing collagen synthesis and hyaluronic acid synthesis. Accordingly, it was confirmed that Dendrobium candidum-derived Saccharomyces cerevisiae KCTC 14268BP, a culture thereof, or a fermentation product thereof has excellent skin improvement effects such as skin moisturizing, wrinkle improvement, elasticity enhancement, promotion of hyaluronic acid production, promotion of collagen synthesis, and anti-aging, etc., as compared to a general yeast, a culture thereof, or a fermentation product thereof.

As used herein, the “skin moisturizing” refers to all of the actions that provide moisture to the skin or block the evaporation of moisture to maintain the skin's flexibility and induce uniform exfoliation to maintain the smooth surface, and “dry” refers to a condition in which such skin moisturizing is not sufficient. Specifically, skin moisturizing may be due to promotion of hyaluronic acid production, but is not limited thereto.

As used herein, the “hyaluronic acid”, a kind of glycosaminoglycans, refers to a chain-like polymeric polysaccharide substance in which glucuronic acid and N-acetylglucosamine residues are repeatedly linked.

As used herein, the “wrinkle” may be largely divided into facial expression wrinkles caused by expression muscles used to make facial expressions and fine wrinkles caused by general or local skin decline. Specifically, wrinkles may be caused by genetic factors, reduction of collagen and elastic fibers present in the skin dermis, external environment, etc. With respect to the objects of the present invention, wrinkles may be used as a meaning including all facial expression wrinkles or fine wrinkles, but are not limited thereto.

As used herein, the “aging” refers to a phenomenon in which biological structures and functions decline over time, and as used herein, the “anti-aging” is not limited to the degree of improvement, as long as aging is improved, such as prevention, suppression, or delay of aging. As used herein, the wrinkle improvement and anti-aging may be due to promotion of collagen synthesis, but are not limited thereto.

The cosmetic composition of the present invention may have a formulation selected from the group consisting of solutions, ointments for external use, creams, foams, nutrient lotions, softening lotions, perfumes, packs, softening water, emulsions, makeup bases, essences, soaps, liquid cleansers, bath preparations, sunscreen creams, sun oil, suspensions, emulsions, pastes, gels, lotions, powders, soaps, surfactant-containing cleansers, oils, powder foundations, emulsion foundations, wax foundations, patches and sprays, but is not limited thereto.

The cosmetic composition of the present invention may further include one or more cosmetically acceptable carriers which are blended in general skin cosmetics, and as common ingredients, for example, oil, water, surfactants, moisturizers, lower alcohols, thickeners, chelating agents, pigments, preservatives, flavoring agents, etc. may be appropriately blended, but are not limited thereto.

The cosmetically acceptable carrier included in the cosmetic composition of the present invention may vary according to the formulation of the cosmetic composition.

When the cosmetic formulation of the present invention is an ointment, a paste, a cream, or a gel, animal oil, vegetable oil, wax, paraffin, starch, tragacanth, cellulose derivative, polyethylene glycol, silicone, bentonite, silica, talc, zinc oxide, etc. may be used as a carrier component, but is not limited thereto. These may be used alone or in a mixture of two or more thereof.

When the formulation of the present invention is a solution or an emulsion, a solvent, solubilizer or emulsifier may be used as a carrier component, for example, water, ethanol, isopropanol, ethyl carbonate, ethyl acetate, benzyl alcohol, benzyl benzoate, propylene glycol, 1,3-butyl glycol oil, etc. may be used. In particular, cottonseed oil, peanut oil, corn germ oil, olive oil, castor oil, sesame oil, glycerol aliphatic esters, polyethylene glycol, and sorbitan fatty acid esters may be used, but are not limited thereto. These carriers may be used alone or in combination of two or more thereof.

When the formulation of the present invention is a suspension, a liquid diluent such as water, ethanol or propylene glycol, a suspending agent such as ethoxylated isostearyl alcohol, polyoxyethyl sorbitol ester and polyoxyethylene sorbitan ester, microcrystalline cellulose, aluminum metahydroxide, bentonite, agar or tragacanth, etc. may be used as a carrier component, but is not limited thereto. These may be used alone or in a mixture of two or more thereof.

When the formulation of the present invention is a soap, alkali metal salts of fatty acid, fatty acid hemiester salts, fatty acid protein hydrolyzate, isethionate, lanolin derivatives, aliphatic alcohol, vegetable oil, glycerol, sugar, etc. may be used as a carrier component, but is not limited thereto. These may be used alone or in a mixture of two or more thereof.

When the formulation of the present invention is a powder or a spray, lactose, talc, silica, aluminum hydroxide, calcium silicate, polyamide powder or a mixture thereof may be used as a carrier component, and in particular, when the formulation is a spray, a propellant such as chlorofluorohydrocarbon, propane/butane, or dimethyl ether may be additionally included.

Meanwhile, all ingredients described in the present invention are preferably included in the composition of the present invention within the range of not exceeding the maximum usage defined in the Regulation on Safety Standards, etc., for Cosmetics, and China's ‘Safety and Technical Standards for Cosmetics’.

Still another aspect of the present invention provides a quasi-drug composition for improving the skin, the quasi-drug composition including any one or more of the Saccharomyces cerevisiae deposited under Accession No. KCTC 14268BP, a culture thereof, and a fermentation product thereof.

The Saccharomyces cerevisiae, culture, fermentation product, and skin improvement are as described above.

As used herein, the term “quasi-drug” may be selected from the group consisting of body cleansers, disinfectant cleaners, detergents, detergents for kitchen, detergents for cleaning, toothpaste, mouth wash, wet wipes, detergents, soaps, hand wash, hair cleaners, hair softeners, humidifier fillers, masks, ointments, and filter filler, but is not limited thereto.

The quasi-drug composition of the present invention may further include a pharmaceutically acceptable carrier, excipient, or diluent, as needed, in addition to the above ingredients. The pharmaceutically acceptable carrier, excipient, or diluent is not limited as long as it does not impair the effects of the present invention, and examples thereof may include fillers, extenders, binders, wetting agents, disintegrants, surfactants, lubricants, sweeteners, fragrances, preservatives, etc.

Representative examples of the pharmaceutically acceptable carrier, excipient, or diluent of the present invention may include lactose, dextrose, sucrose, sorbitol, mannitol, xylitol, maltitol, starch, gelatin, glycerin, gum acacia, alginate, calcium phosphate, calcium carbonate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, propylene glycol, polyethylene glycol, vegetable oil, injectable esters, Witepsol, Macrogol, Tween 61, cacao butter, laurin butter, etc.

When the strain of the present invention, a culture thereof, or a fermentation product thereof is used as a quasi-drug, one or more active ingredients exhibiting the same or similar function may be further included. For example, known components for enhancement of skin beneficial bacteria, suppression of skin harmful bacteria, skin soothing, improvement of skin inflammation, skin whitening, skin regeneration, wound healing, skin moisturizing, anti-aging, and elasticity enhancement may be included. When additional components for wrinkle improvement, skin moisturizing, anti-aging, and elasticity enhancement are included, the effects of wrinkle improvement, skin moisturizing, anti-aging, and elasticity enhancement of the composition of the present invention may be further increased. When the above components are added, skin safety, ease of formulation, and stability of active ingredients may be considered according to combination use.

The quasi-drug composition of the present invention may further include one or two or more components selected from the group consisting of retinoic acid, TGF, animal placenta-derived proteins, betulinic acid, and a chlorella extract as a skin anti-aging component known in the art; non-steroidal flufenamic acid, ibuprofen, benzydamine, indomethacin, prednisolone, dexamethasone, allantoin, azene, hydrocortisone as an anti-inflammatory components known in the art; derivatives thereof and various plant extracts. Additional components may be included in an amount of 0.0001% by weight to 10% by weight with respect to the total weight of the composition, and the above range of content may be adjusted according to requirements such as skin safety, ease of formulation of the strain or the fermentation product of the present invention, etc.

The formulation method, dosage, usage method, components, etc. of the quasi-drug may be appropriately selected from common techniques known in the art.

Still another aspect of the present invention provides a food composition for improving the skin, the food composition including any one or more of the Saccharomyces cerevisiae deposited under Accession No. KCTC 14268BP, a culture thereof, and a fermentation product thereof.

The Saccharomyces cerevisiae, culture, fermentation product, and skin improvement are as described above.

As used herein, the term “food” may include meat, sausage, bread, chocolate, candy, snacks, confectionery, pizza, ramen, other noodles, gum, and dairy products including ice cream, various soups, beverages, teas, drinks, alcoholic beverages, vitamin complexes, health functional foods, and health foods, and includes all foods in the ordinary sense.

The health functional food (functional food) is the same term as food for special health use (FoSHU), and refers to a food having high medicinal and medical effects, which is processed to effectively exert a body-regulating function as well as to supply nutrients. Here, the term “functional” means that it is taken for the purpose of controlling nutrients with respect to structures and functions of the human body or of obtaining effects beneficial for health care, such as physiological effects. The food of the present invention may be prepared by a method commonly used in the art, and may be prepared by adding raw materials and ingredients which are commonly added in the art. In addition, the formulation of the food may be prepared without limitation, as long as it is acceptable as food. The food composition of the present invention may be prepared as various types of formulations. Unlike general drugs, the food composition includes a food as a raw material, and therefore, it has advantages of being free from side effects that may occur when taken for a long period of time, and of being highly portable, and therefore, the food of the present invention may be ingested as an auxiliary to enhance the effect of improving the skin.

The health food refers to food which brings about an active health-preserving or enhancing effect, as compared to the common food, and the health supplement food refers to food to be ingested for the purpose of health supplementation. In some cases, the terms of health functional food, health food, and health supplement food are used interchangeably.

Specifically, the health functional food is food prepared by adding any one or more of the Saccharomyces cerevisiae deposited under Accession No. KCTC 14268BP, a culture thereof, and a fermentation product thereof to a food material such as beverage, tea, spice, gum, chips, etc., or by encapsulation, powdering, or suspension, and brings about a specific effect to health when consumed. However, unlike general drugs, it has advantages of being free from side effects that may occur when a drug is administered for a long period of time because it is prepared by using food as raw materials.

Since it is possible to routinely ingest the food composition of the present invention, it may be very useful because a high skin improvement effect may be expected.

The composition may further include a physiologically acceptable carrier, and the type of carrier is not particularly limited and any carrier may be used as long as it is commonly used in the art.

Further, the composition may further include additional ingredients which are commonly used in food compositions to improve odor, taste, vision, etc. For example, it may include vitamins A, C, D, E, B1, B2, B6, B12, niacin, biotin, folate, panthotenic acid, etc. Furthermore, it may include minerals such as zinc (Zn), iron (Fe), calcium (Ca), chromium (Cr), magnesium (Mg), manganese (Mn), copper (Cu), etc. In addition, it may include amino acids such as lysine, tryptophan, cysteine, valine, etc.

Moreover, the composition may include food additives such as preservatives (potassium sorbate, sodium benzoate, salicylic acid, sodium dehydroacetate, etc.), disinfectants (bleaching powder and high-grade bleaching powder, sodium hypochlorite, etc.), antioxidants (butylhydroxyanisole (BHA), butylhydroxytoluene (BHT), etc.), coloring agents (tar color, etc.), color former agents (sodium nitrite, etc.), bleaching agents (sodium sulfite, etc.), seasoning (sodium glutamate (MSG), etc.), sweeteners (dulcin, cyclamate, sodium, saccharin, etc.), flavoring agents (vanillin, lactones, etc.), swelling agents (alum, potassium hydrogen D-tartrate), reinforcing agents, emulsifying agents, thickener (thickening agents), coating agents, gum bases, foam inhibitors, solvents, enhancers, etc. These additives may be selected and used in an appropriate amount according to the type of food.

Any one or more of the Saccharomyces cerevisiae deposited under Accession No. KCTC 14268BP, the culture thereof, and the fermentation product thereof may be added as it is or used together with other food or food ingredients, and may be appropriately used according to a common method. The mixing ratio of the active ingredients may be appropriately determined according to the purpose thereof (prevention, health, or therapeutic treatment). In general, during the production of food or beverage, the food composition of the present invention may be added in an amount of 50 parts by weight or less, specifically 20 parts by weight or less with respect to the food or beverage, but is not limited thereto.

As an example of the food composition of the present invention, it may be used as a health beverage composition, and in this case, various flavoring agents or natural carbohydrates may be added as additional ingredients as in the case of common beverages. The above-mentioned natural carbohydrates may include monosaccharides such as glucose and fructose; disaccharides such as maltose and sucrose; polysaccharides such as dextrin and cyclodextrin; and sugar alcohols such as xylitol, sorbitol, erythritol, etc. Sweeteners may include natural sweeteners such as thaumatin and stevia extract; and synthetic sweeteners such as saccharin, aspartame, etc. A proportion of the natural carbohydrate is generally about 0.01 g to 0.04 g, specifically about 0.02 g to 0.03 g per 100 mL of the composition of the present invention.

In addition to the above, the health beverage composition may include various nutritional supplements, vitamins, electrolytes, flavoring agents, coloring agents, pectic acid, salts of pectic acid, alginic acid, salts of alginic acid, organic acids, protective colloid thickening agents, pH adjusters, stabilizers, preservatives, glycerin, alcohol, or carbonating agents, etc. In addition, it may include a flesh for the production of natural fruit juices, fruit juice drinks or vegetable drinks. These ingredients may be used independently or in a mixture. The proportion of such additives is not critical, but is generally selected in the range of 0.01 part by weight to 0.1 part by weight based on 100 parts by weight of the composition of the present invention.

The food composition of the present invention may be included in various weight percentages as long as it may exhibit the skin improvement effect. Specifically, it may include any one or more of the Saccharomyces cerevisiae deposited under Accession No. KCTC 14268BP, the culture thereof, and the fermentation product thereof in an amount of 0.00001% by weight to 100% by weight with respect to the total weight of the food composition.

The Saccharomyces cerevisiae deposited under Accession No. KCTC 14268BP, the culture thereof, and the fermentation product thereof may be included in the quasi-drug, food, and/or cosmetic composition, as described above, but may also be included in a pharmaceutical composition.

Still another aspect of the present invention provides a method of improving the skin, the method including administering, to an individual, any one or more of the Saccharomyces cerevisiae strain deposited under Accession No. KCTC 14268BP, the culture thereof, and the fermentation product thereof; or the food composition including the same.

The administering may be oral administering, and may be ingestion and/or inhalation, and the Saccharomyces cerevisiae, the culture, the fermentation product, and the skin improvement are as described above.

In the present invention, the term “individual” refers to all animals including mammals such as mice, livestock, and humans, etc., but is not limited thereto.

Still another aspect of the present invention provides a method of improving the skin, the method including applying, to an individual's skin, any one or more of the Saccharomyces cerevisiae strain deposited under Accession No. KCTC 14268BP, the culture thereof, and the fermentation product thereof; or the cosmetic composition including the same; and/or the quasi-drug composition including the same.

The Saccharomyces cerevisiae, the culture, the fermentation product, and the skin improvement are as described above.

As used herein, the term “applying” means bringing the composition of the present invention into contact with an individual's skin by any suitable method, and includes all of the actions aimed at absorbing the corresponding composition into the skin through this, but is not limited thereto.

Still another aspect of the present invention provides use of any one or more of the Saccharomyces cerevisiae strain deposited under Accession No. KCTC 14268BP, the culture thereof, and the fermentation product thereof, for the skin improvement.

The Saccharomyces cerevisiae, the culture, the fermentation product, and the skin improvement are as described above.

### MODE FOR CARRYING OUT THE INVENTION

Hereinafter, the present invention will be described in more detail with reference to Examples. However, these Examples and Experimental Example are for illustrative purposes only, and the scope of the present invention is not intended to be limited by these Examples and Experimental Example.

### Example 1: Isolation and Selection of Yeast Strains from Dendrobium candidum

1-1: Yeast Isolation from Dendrobium candidum

Dendrobium candidum flowers were put into YM broth and cultured under shaking at 30° C. for 4 days, inoculated into a solid medium, and cultured at 30° C. for 3 days. Colonies formed on a solid medium were isolated and identified. At this time, 187 types of strains presumed to be yeast (the genus Saccharomyces) were isolated, followed by microorganism identification.

As a result, 6 types of strains classified as Saccharomyces cerevisiae and 1 type of strain classified as Saccharomyces boulardii were obtained.

7 types of yeasts (6 types of Saccharomyces cerevisiae and 1 type of Saccharomyces boulardii) obtained in the present invention are shown in Table 1 below.

TABLE 1

| NO | Microorganism |
|---|---|
| 1 | Saccharomyces cerevisiae #1-7 |
| 2 | Saccharomyces cerevisiae #2-13 |
| 3 | Saccharomyces cerevisiae #3-5 |
| 4 | Saccharomyces cerevisiae #4-17 |
| 5 | Saccharomyces cerevisiae #5-31 |
| 6 | Saccharomyces boulardii #6-21 |
| 7 | Saccharomyces cerevisiae #7-3 |

1-2: Selection of Yeast Isolated from Dendrobium candidum

In order to select strains that are easy to use because of the low off-flavor among 7 types of yeast fermentation products, 7 types of the yeast fermentation products were prepared, and then 10 test subjects were subjected to an off-flavor test for the respective yeast fermentation products. The indexes of the off-flavor test are shown in Table 2 below.

TABLE 2

| Score | Index |
|---|---|
| 1 | No flavor |
| 2 | Undistinguishable as to whether there is a distinctive flavor or not |
| 3 | Distinctive flavor |
| 4 | Distinctive and unpleasant flavor |
| 5 | Distinctive and very unpleasant flavor |

As a result, as shown in Table 3, it was confirmed that No. 5 exhibited the least off-flavor.

TABLE 3

| Strain | Score (Average of scores from the 10 subjects) |
|---|---|
| 1 | 2.9 |
| 2 | 3.5 |
| 3 | 3.1 |
| 4 | 2.8 |
| 5 | 1.5 |
| 6 | 3.0 |
| 7 | 2.6 |

The Dendrobium candidum-derived yeast Saccharomyces cerevisiae #5-31 strain obtained in the present invention was deposited under the Budapest Treaty in the international depository authority, Korean Culture Center of Microorganisms on Aug. 10, 2020, with the Accession No. KCTC 14268BP.

### Example 2: Preparation of Fermentation Product of Dendrobium candidum-Derived Yeast

The Dendrobium candidum-derived yeast (Saccharomyces cerevisiae KCTC 14268BP) strain was inoculated into an YPD medium, cultured under shaking at 30° C. for 4 days, and cells were separated by centrifugation. The cells were suspended in purified water, and disrupted by sonication, and finally, a fermentation product of Dendrobium candidum-derived yeast was prepared. Specific conditions and configurations are shown in Table 4 below.

TABLE 4

| | |
|---|---|
| Strain | Saccharomyces cerevisiae #5-31 strain |
| | (KCTC 14268BP) |
| Culture conditions | YPD media, 4 day, 30° C. 180 rpm |
| Sonication | Amplitude 30° C., Process time 40 min, |
| | Pulse-ON time 1 S, Plus-OFF time 4 S |
| Concentration of culture | 5% |
| extract of Dendrobium | |
| candidum-derived yeast | |

### Example 3: Examination of Collagen Synthesis Enhancement Effect of Fermentation Product of Dendrobium candidum-Derived Yeast

Human dermal fibroblasts were treated with each sample for 48 hours. Thereafter, a collagen synthesis rate was measured by detecting collagen using a method of a procollagen type I C-peptide (PIP) EIA kit. The collagen synthesis efficiency of the sample was expressed as an increase rate (%) of collagen synthesis, which was calculated and quantified in comparison to a negative control group (DMEM containing 0% serum).

The calculation formula of the increase rate of collagen synthesis, each sample, and the results thereof are shown in Table 5 below.

Increase rate of collagen synthesis (%)=(Collagen synthesis rate of Sample−Collagen synthesis rate of Negative control)/Collagen synthesis rate of Negative control

TABLE 5

| | Increase rate of |
|---|---|
| Sample | collagen synthesis |
| Normal cell (negative control) | 0 |
| Normal cell + TGF-β (positive control, 10 ppb) | 15.2 |
| Normal cell + fermentation product of Dendrobium | 38.3 |
| candidum-derived yeast (KCTC 14268BP) (0.01%) | |
| Normal cell + fermentation product of normal yeast | 6.0 |
| (KCTC 7296) (0.01%) | |

As shown in the results of Table 5, it was confirmed that collagen synthesis ability increased when normal cells were treated with the fermentation product of Dendrobium candidum-derived yeast (KCTC 14268BP). In this regard, as compared to the fermentation product of the normal yeast (KCTC 7296) prepared under the same conditions, the fermentation product of Dendrobium candidum-derived yeast (KCTC 14268BP) had the high increase rate.

Accordingly, it was found that the fermentation product of the Dendrobium candidum-derived yeast of the present invention exhibits excellent effects in terms of skin wrinkle improvement, elasticity enhancement, and anti-aging.

### Example 4: Examination of Hyaluronic Acid Synthesis Enhancement Effect of Fermentation Product of Dendrobium candidum-Derived Yeast

Human keratinocytes (HaCaT) were treated with each sample for 72 hours. Thereafter, a hyaluronic acid synthesis rate was derived by measuring hyaluronic acid using R&D systems' Quantikine ELISA Hyaluronan. The skin moisturizing ability of the sample was expressed as an increase rate (%) of hyaluronic acid synthesis, which was calculated and quantified in comparison to a negative control group (treated with DMEM containing 0% serum).

The calculation formula of the increase rate of hyaluronic acid synthesis, each sample, and the results thereof are shown in Table 6 below.

Increase rate of hyaluronic acid synthesis (%)=(Hyaluronic acid synthesis rate of Sample−Hyaluronic acid synthesis rate of Negative control)/Hyaluronic acid synthesis rate of Negative control

TABLE 6

| | Increase rate |
|---|---|
| | of hyaluronic |
| Sample | acid synthesis |
| Normal cell (negative control) | 0 |
| Normal cell + Retinoic acid (positive control, 1 ppm) | 13.8 |
| Normal cell + fermentation product of Dendrobium | 9.8 |
| candidum-derived yeast (KCTC 14268BP) (0.01%) | |
| Normal cell + fermentation product of normal yeast | 9.3 |
| (KCTC 7296) (0.01%) | |

As shown in the results of Table 6, it was confirmed that hyaluronic acid synthesis ability increased when normal cells were treated with the fermentation product of Dendrobium candidum-derived yeast (KCTC 14268BP). In this regard, as compared to the fermentation product of the normal yeast (KCTC 7296) prepared under the same conditions, the fermentation product of Dendrobium candidum-derived yeast had the high increase rate.

Accordingly, it was found that the fermentation product of the Dendrobium candidum-derived yeast (KCTC 14268BP) of the present invention exhibits excellent effects in terms of skin moisturizing.

Based on the above description, it will be understood by those skilled in the art that the present invention may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1.-9. (canceled)

10. A method of improving the skin, the method including: administering, to an individual, any one or more of the Saccharomyces cerevisiae strain deposited under Accession No. KCTC 14268BP, the culture thereof, and the fermentation product thereof.

11. The method of claim 10, wherein the skin improvement is any one or more selected from the group consisting of skin moisturizing, wrinkle improvement, elasticity enhancement, promotion of hyaluronic acid production, promotion of collagen synthesis, and anti-aging.

12. A method of improving the skin including: applying, to an individual's skin, any one or more of the Saccharomyces cerevisiae strain deposited under Accession No. KCTC 14268BP, the culture thereof, and the fermentation product thereof.

13. The method of claim 12, wherein the skin improvement is any one or more selected from the group consisting of skin moisturizing, wrinkle improvement, elasticity enhancement, promotion of hyaluronic acid production, promotion of collagen synthesis, and anti-aging.
